(19) United States
(12) Patent Application Publication
(10) Pub. No.: US 2026/0028625 A1
(43) Pub. Date: Jan. 29, 2026

(54) PRODUCT PREPARATION BASED ON APPLICATION OF SGRNA FOR THE TREATMENT OF HUNTINGTON'S DISEASE

(71) Applicants: Chenjian LI, Irvine, CA (US); Sushuang ZHENG, Beijing (CN)

(72) Inventors: Chenjian LI, Irvine, CA (US); Sushuang ZHENG, Beijing (CN)

(21) Appl. No.: 19/105,196

(22) PCT Filed: Mar. 30, 2023
(86) PCT No.: PCT/US2023/010120

§ 371 Date: Feb. 20, 2025

(30) Foreign Application Priority Data
Aug. 22, 2022 (CN) 202211005017.7

(51) Int. Cl. C12N 15/113; A61K 31/7105; A61K 38/46; A61K 48/00; A61P 25/28; C12N 9/22; C12N 15/86

(52) U.S. Cl. CPC C12N 15/113; A61K 31/7105; A61K 38/465; A61K 48/005; A61K 48/0075; A61P 25/28; C12N 9/226; C12N 15/86; C12N 2310/20; C12N 2750/14143; C12N 2750/14152

(57) ABSTRACT

The present disclosure relates to an sgRNA and its application in the preparation of a product for the treatment of Huntington's disease. The present disclosure was designed and screened to obtain an sgRNA targeting exon 1 of the human HTT gene as shown in SEQ ID NO: 1 or SEQ ID NO: 2. The CRISPR/Cas9 system mediated HTT gene knockout strategy based on this sgRNA and its high homologue sgRNA can efficiently knock out the human Huntingtin gene and achieve gene therapy for Huntington's disease.

### FIELD OF THE DISCLOSURE

The present disclosure relates in general to the technical field of disease treatment, and more particularly, to an sgRNA and its application in the preparation of products for the treatment of Huntington's disease.

### BACKGROUND OF THE DISCLOSURE

Huntington's Disease (HD) is an autosomal dominant neurodegenerative disorder associated with an unstable repeat expansion (>36 repeats) of the CAG trinucleotide in exon 1 of the huntingtin gene (HTT), expressing a mutant huntingtin protein (mHTT) with gain-of-function neurotoxic effects. Huntington's disease is invariably a neurodegenerative disorder due to the HTT gene, and therefore any pathogenic process caused by this genetic factor could be a potential therapeutic target. The current investigation of therapeutic modalities for HD follows this process. In HD, CAG trinucleotide repeats overexpansion in exon 1 of the HTT gene leads to ubiquitous expression of mutant Huntington protein. mHTT is thought to be a major cause of HD toxicity, and therefore its pathogenic process can be slowed down by directly reducing mHTT expression.

Gene therapy refers to an emerging therapeutic approach by modifying or manipulating the expression of genes to alter the biological properties of cells. Gene therapy acts directly on the genetic material and has three main mechanisms of action: (1) replacement: replacing the disease-causing gene with a normal gene; (2) inactivation: inactivating an abnormal gene; and (3) insertion: introducing a new or modified gene into cells. Studies on therapeutic tools affecting mHTT expression have developed rapidly in recent years. For example, gene silencing strategies, include RNA interference (RNAi) using siRNA or microRNA, and antisense oligonucleotide-based gene silencing (ASOs), that act on mRNA to inhibit Huntingtin protein synthesis. In addition, DNA editing using zinc-finger nuclease (ZFN) and CRISPR-Cas9 technology are also included to suppress Huntingtin gene expression.

ASOs and RNAi complexes can selectively bind to mRNA through Watson-Crick base pairing, thus triggering the RNA degradation mechanism. ASOs are synthetic single-stranded DNA molecules that bind to the pre-mRNA of target genes in the nucleus and catalyze their degradation by RNase H. RNAi is an RNA-based gene silencing technology, including siRNA (small interfering RNA), shRNA (short hairpin RNA) and miRNA (microRNA), which can bind to mature mRNA in the cytoplasm and be degraded by RNA-induced silencing complex (RISC). ASOs are different from RNAi in that they act on different target genes at different sites. RNAi acts on the spliced mRNA and can only target exons. However, ASOs are more flexible in sequence selection, as they can interact with pre-mRNA, thereby targeting both exons and introns. Single-stranded DNA is more diffusible in the central nervous system and can be taken up by neurons and other cells, so ASOs injected into mouse or mammalian cerebrospinal fluid can be widely diffused in the brain. However, double-stranded RNAs have low diffusion capacity in the central nervous system (CNS) and are not effectively absorbed by cells, so in most cases, RNAi should be delivered via viral vectors and require localized injection into the brain parenchyma. Although this drug delivery approach is more complex, it can achieve lifelong treatment with a single drug injection. Studies on ASOs-and RNAi-based treatments for Huntington's disease have made some progress in several in vitro and in vivo animal models, but none of these studies has yet been translated into successful clinical trial.

Directly targeting DNA to suppress mutant Huntingtin gene transcription seems more promising than targeting RNA, and the design and use of gene editing technologies to treat Huntington's disease, while more challenging, can in principle improve all aspects of

HD, including non-ATG initiated translation, variable splicing and other mechanisms that may occur, which are more difficult to address with RNA-lowering down therapies. The main HD gene therapies currently being investigated that target DNA include zinc finger nuclease (ZFN) and CRISPR-Cas9, both in preclinical studies. CRISPR-Cas9 enables the editing of targeted genes using Cas9 nucleases with guide RNA. When both Cas9 protein and single-stranded guide RNA (sgRNA) are expressed in cells, sgRNA binds to specific sequences on the genome, recruiting Cas9 protein and generating DNA double-strand breaks that activate the DNA double-strand break repair mechanism. Sequence insertions or deletions (Indels) are introduced near the damage site, resulting in appearance of pre-mature stop codon and gene inactivation. Genome editing using CRISPR-Cas9 is a rapidly growing field with great potential for the study and treatment of diseases including Huntington's disease.

However, the current CRISPR/Cas-based HTT knockout strategies still suffer from low gene editing efficiency, limited improvement in disease deficits, and short time-scale investigation for treatment effects.

### SUMMARY OF THE DISCLOSURE

It is therefore an object of the present disclosure to provide an sgRNA for CRISPR/Cas system with high efficiency of HTT gene knockout, significant improvement of disease phenotypes, and long-term follow-up of treatment effect duration.

The sgRNA has a nucleotide sequence that includes nucleotide sequence as shown in SEQ ID NO: 1 or SEQ ID NO: 2, or a nucleotide sequence having at least 95% homology with the nucleotide sequence shown by SEQ ID NO: 1 or SEQ ID NO: 2, and having the same function or a nucleotide sequence obtained from the nucleotide sequence shown in SEQ ID NO: 1 or SEQ ID NO: 2 by a deletion, substitution or addition of 1 to 6 bases and having the same function.

In one of the embodiments, the nucleotide sequence of the sgRNA is a nucleotide sequence having at least 98% homology with the nucleotide sequence shown in SEQ ID NO: 1 or SEQ ID NO: 2 and having the same function.

In one of the embodiments, the nucleotide sequence of the sgRNA is a nucleotide sequence obtained by a deletion, substitution or addition of 1 to 3 bases at the 5′ end or 3′ end of the nucleotide sequence shown in SEQ ID NO: 1 or SEQ ID NO: 2, and having the same function.

The present disclosure also provides a DNA fragment, which encodes the sgRNA as described above, and a recombinant expression vector, with the recombinant expression vector including a DNA fragment as described above.

In one of the embodiments, the recombinant expression vector further includes a fragment of a sequence encoding a Cas nuclease.

In one of the embodiments, the Cas endonuclease coding sequence is a SaCas9 endonuclease coding sequence, a SpCas9 endonuclease coding sequence, a Cas12a endonuclease coding sequence, a Cas12b endonuclease coding sequence, a Cas12e endonuclease coding sequence, a Cas12j endonuclease coding sequence, a Cas12f1 endonuclease coding sequence, a Cas13a endonuclease coding sequence, or a Cas14a endonuclease coding sequence.

In one of the embodiments, the recombinant expression vector is a lentiviral vector, an adenovirus vector, an adeno-associated virus vector, a herpesvirus vector, a poxvirus vector, a baculovirus vector, a papillomavirus vector, or a papillomavirus vector. In one embodiment, the recombinant expression vector is an AAV9 viral vector.

The present disclosure also provides a virus, the virus having a genome containing a nucleotide sequence encoding the sgRNA as described above.

In one of the embodiments, the virus is a lentivirus, adenovirus, adeno-associated virus, herpesvirus, poxvirus, baculovirus, papillomavirus or papillary polyomavirus.

In one of the embodiments, the virus is an AAV9 virus.

The disclosure also provides a host cell, which has a genome containing a DNA fragment or recombinant expression vector as described above.

In one of the embodiments, the host cell is a CHO cell, COS cell, NSO cell, HeLa cell, BHK cell or HEK293T cell.

The present disclosure also provides for the use of sgRNA, DNA fragments, recombinant expression vectors, viruses or host cells as described above in the preparation of products for the treatment of Huntington's disease.

In one of the embodiments, the product is a reagent, kit, drug or device.

The present disclosure also provides a drug for the treatment of Huntington's disease comprising a sgRNA, DNA fragment, recombinant expression vector, virus, or host cell as described above, and a pharmaceutically usable excipient.

In one of the embodiments, the dosage form of the drug is an injection.

In one of the embodiments, the excipient comprises one or more of a diluent, a preservative, buffer, disintegrant, antioxidant, suspension aid, colorant and excipient.

The present disclosure also provides an HTT knockout method that includes the steps of: exogenously expressing sgRNA as described above in a target cell, and having a Cas endonuclease.

The present disclosure also provides a method of treating Huntington's disease that includes the steps of delivering a Cas endonuclease system and sgRNA as described above into striatum and cortical regions of a brain of a diseased individual.

In one of the embodiments, the delivery is a stereotactic injection of the brain.

The present disclosure has been screened to obtain an sgRNA targeting exon 1 of the human-derived HTT gene with a nucleotide sequence as shown in SEQ ID NO: 1 or SEQ ID NO: 2. The CRISPR/Cas9-mediated HTT gene knockout strategy based on this sgRNA and its higher homology sgRNA can efficiently knock out the human-derived Huntingtin gene and achieve gene therapy for Huntington's disease. In experimental tests, the delivery of SaCas9 endonuclease and the aforementioned sgRNA targeting exon 1 of the human HTT gene into both striatal and cortical regions of the brain via the AAV9 vector resulted in an approximately 90% reduction in HTT protein expression, particularly at the individual level, which significantly improved HD-related phenotypic defects. This gene therapy strategy has great potential to provide new ideas and techniques for the treatment of Huntington's disease.

### BRIEF DESCRIPTION OF THE DRAWINGS

FIG. 1 a schematic structural diagram of the AAV9-SaCas9-HTT sgRNA vector according to the exemplary embodiment of the present disclosure that includes the U6 promoter-driven sgRNA and CMV promoter-driven SaCas9 inserted into the AAV vector with a sequence length of 4.5 kb between the two ITRs; ITR: inverted terminal repeat; NLS: nuclear localization signal sequence; and 3×HA: three tandem repeats of the human influenza hemagglutinin (HA) tag.

FIGS. 2A and 2B illustrates the results of the sgRNA editing efficiency of the HTT gene in HEK293T cells according to the embodiment of the present disclosure, where FIG. 2A is a fluorescence micrograph (scale bar: 100 μm) of HEK293T cells in which co-transfection of pEGFPc3-HTT-exon1-20Q/120Q with AAV-SaCas9-sgRNA reduced Huntingtin protein expression and aggregation. FIG. 2B is a quantitative analysis of the fluorescence intensity in FIG. 2A. Data are analyzed by one-way ANOVA with Tukey's post hoc test, data are presented as mean±SEM, from left to right, data for Control sgRNA, hHTT sgRNA1 and hHTT sgRNA2, where **p<0.01, ***p<0.001 (N=4 for Control sgRNA in the 20 CAG Repeats group, N=3 in the hHTT sgRNAl and hHTT sgRNA2 groups, and N=3 in each of the 120 CAG Repeats groups).

FIGS. 3A-3D illustrates the effect of protein immunoblotting assay to detect the editing of HTT gene by sgRNA according to the exemplary embodiment of the present disclosure, where FIG. 3A shows the transfection of sgRNA1 and sgRNA2 in HEK293T cells exogenously expressing HTT-exon1-20Q with GFP tags, and the expression levels of SaCas9 and exon 1-20Q were detected by immunoblotting assay using anti-HA antibody and anti-GFP antibody (β-actin is the protein internal reference). FIG. 3B shows the quantitative analysis of GFP signal (left panel) and HA signal (right panel) levels in FIG. 3A, from left to right, Control sgRNA, hHTT sgRNA1 and hHTT sgRNA2, respectively. In anti-GFP, the GFP expression level of the sgRNA1 group relative to the control group was 0.2844±0.1551 (p=0.0087), the expression level of the sgRNA2 group relative to the control group was 0.5706±0.1099 (p=0.0727). In anti-HA, the expression level of SaCas9 in the sgRNAl group was 0.8814+0.1487 (p=0.7178), and the expression level in the sgRNA2 group was 0.9048±0. 0.1049 (p=0.8040). FIG. 3C is the transfection of sgRNA1 and sgRNA2 in HEK293T cells exogenously expressing GFP-tagged HTT-exon1-120Q, using Western blot experiments to detect SaCas9 and exon1-the expression level of 120Q (β-actin as control). FIG. 3D is the quantitative analysis of the GFP signal (left panel) and HA signal (right panel) levels in FIG. 3C. From left to right, are data for control sgRNA, hHTT sgRNA1 and hHTT sgRNA2. In anti-GFP, the expression level of GFP in the sgRNA1 group relative to the control group was 0.0688 (p=0.0069), the expression level in the sgRNA2 group relative to the control group was 0.7238±0.1447 (p=0.1675); and in anti-HA, the expression level of SaCas9 in the sgRNA1 group relative to the control group was 1.162±0.093 (p=0.6217), the expression level in the sgRNA2 group relative to the control group was 0.9782±0.1835 (p=0.9907). Data are analyzed by one-way ANOVA with Tukey's post hoc test, where ** indicates p<0.01, N=3 in each group of experiments.

FIG. 4 illustrates the expression pattern of AAV9-GFP in primary motor cortex and striatum according to the embodiment of the present disclosure, where M1 is the primary motor cortex; M2 is the secondary motor cortex; CC is the corpus callosum; LV is the lateral ventricle; and Str is the striatum (scale bar: 500 μm).

FIG. 5 illustrates the protein expression level of mHTT in the striatum, cortex and cerebellum of 4-month-old, 7-month-old and 11-month-old BAC226Q-HTTg1 mice according to the embodiment of the present disclosure, where the left panel shows the immunoblotting reaction to detect mHTT protein expression in the striatum, cortex and cerebellum of 4-month-old and 7-month-old Non tg-SaCas9, BAC226Q-SaCas9 and BAC226Q-HTTg1 mice. AAV9-SaCas9 or AAV9-SaCas9-HTTg1 was injected into the striatum and cortex of mice only, and not in the cerebellum; The right panel shows the expression of mHTT protein in the striatum of 11-month-old uninjected wild-type mice (Non tg-w/o inj.), non tg-SaCas9, BAC226Q-SaCas9 and BAC226Q-HTTg1 mice by immunoblotting (mHTT protein was detected by 1C2 antibody, β-actin was the internal control).

FIGS. 6A-6D illustrates the signal of mHTT in the striatum (top) and primary motor cortex (bottom) of 4-month-old mice detected by S830 antibody according to the embodiment of the present disclosure, where FIG. 6A shows immunofluorescence staining of mHTT signals in the striatum of 4-month-old Non tg-SaCas9, HD-SaCas9 (BAC226Q-SaCas9) and HD-HTTg1 (BAC226Q-HTTg1) mice, with mHTT labeled with S830 antibody, and the nucleus labeled with the nuclear stain DAPI. Al and A2 in the rightmost column are enlarged images of the corresponding regions on the left 50 (scale bar: 50 μm); FIG. 6B shows the quantitative analysis of striatum mHTT aggregation signal in FIG. 6A, From left to right, are data for Non tg-SaCas9, HD-SaCas9 and HD-HTTg1. The aggregation signal of mHTT is analyzed by Analyze particles in Image J (aggregation signal=aggregate counts× integrated density); FIG. 6C is immunofluorescence staining of mHTT signals in primary motor cortex of 4-month-old Non tg-SaCas9, HD-SaCas9 (BAC226Q-SaCas9), and HD-HTTg1 (BAC226Q-HTTg1) mice; C1 and C2 in the rightmost column are enlarged images of the corresponding regions on the left (scale bar: 50 μm); and FIG. 6D is statistics of mHTT aggregation signals in primary motor cortex in FIG. 6C, from left to right, are data for Non tg-SaCas9, HD-SaCas9, and HD-HTTg1. Data are analyzed by one-way ANOVA with Tukey's post hoc test, where *** indicates p<0.001, ** indicates p<0.01, * indicates p<0.05, and N=5 in each group of experiments.

FIGS. 7A-7D illustrates the signal of mHTT in the striatum (top) and primary motor cortex (bottom) of 7-month-old mice detected by S830 antibody in one embodiment of the present disclosure; where FIG. 7A shows immunofluorescence staining of mHTT signals in the striatum of 7-month-old Non tg-SaCas9, HD-SaCas9 (BAC226Q-SaCas9) and HD-HTTg1 (BAC226Q-HTTg1) mice, with mHTT labeled using S830 antibody. The nuclei were labeled with the nuclear stain DAPI, the rightmost columns A1 and A2 are enlarged images of the corresponding regions on the left (scale bar: 50 μm), and FIG. 7B shows the statistics of mHTT signals in the striatum in FIG. 7A. The mHTT signals were counted as aggregation signals using the analyze particles function in Image J; FIG. 7C shows the immunofluorescence staining of mHTT signals in primary motor cortex of 7-month-old Non tg-SaCas9, HD-HTTg1 (BAC226Q-HTTg1) and HD-HTTg1 (BAC226Q-HTTg1) mice; C1 and C2 in the rightmost column are enlarged images of the corresponding areas on the left (scale bar: 50 μm); and FIG. 7D shows the statistics of mHTT signals in the primary motor cortex in FIG. 7C. Data are analyzed by one-way ANOVA with Tukey's post hoc test, where *** indicates p<0.001, ** indicates p<0.01, * indicates p<0.05, and N=5 in each group of experiments.

FIGS. 8A and 8B illustrates the mHTT signal in the brain of 11-month-old mice

detected by S830 antibody according to the embodiment of the present disclosure, where FIG. 8A shows immunohistochemical staining of the mHTT signal in the brains of 11-month-old BAC226Q-GFP (left) and BAC226Q-HTTg1 (right) mice injected with AAV9-SaCas9-HTTg1 or AAV9-GFP as controls, and the mHTT signal was labeled with S830 antibody, and the images were obtained by whole brain slice scanning (scale bar: 2000 μm). FIG. 8B is a magnified image corresponding to the light gray box (left) and dark gray box (right) in FIG. 8A, showing the mHTT staining signal in the striatum.

FIG. 9 illustrates the phenotypic results of AAV-SaCas9-HTTg1 according to the embodiment of the present disclosure with significantly enhanced phenotype of BAC226Q mice in the rota-rod test; behavioral performance of 11-week-old to 16-week-old Non tg-SaCas9, BAC226Q-SaCas9 and BAC226Q-HTTgmice on uniformly accelerated rota-rod, and recording the time the mice remained on the rotarod (latency to fall). Data are analyzed by two-way ANOVA with Bonferroni's post hoc test, where *** indicates p<0.001, ** indicates p <0.01, * indicates p<0.05, data are presented as mean+SEM; N=9 for BAC226Q-HTTg1 group, N=10 for BAC226Q-SaCas9 group, and N=10 for Non tg-SaCas9 group.

FIGS. 10A-10E illustrates the phenotypic results of AAV-SaCas9-HTTg1 rescued BAC226Q mice in the gait analysis according to the embodiment of the present disclosure, where FIG. 10A is a representative footprint of 6-month-old Non tg-SaCas9 (top), BAC226Q-SaCas9 (middle) and BAC226Q-HTTg1 mice (bottom) in the gait analysis; FIG. 10B is the gait regularity index of 2.5-, 4- and 6-month-old Non tg-SaCas9, BAC226Q-SaCas9 and BAC226Q-HTTg1 mice; FIG. 10C, FIG. 10D and FIG. 10E are the footprint area of 2.5-, 4- and 6-month-old Non tg-SaCas9, BAC226Q-SaCas9 and BAC226Q-HTTg1 mice, respectively. Data are analyzed by one-way ANOVA with Tukey's post hoc test, where *** indicates p<0.001, ** indicates p<0.01, * indicates p<0.05, data are presented as mean±SEM, and N=10 for all other groups except for N=9 for 2.5-and 4-month-old BAC226Q-HTTg1 group.

FIGS. 11A-11D illustrates the behavioral data of mice in the open field test according to the embodiment of the present disclosure, where FIG. 11A is a heat map showing the position and time of 6-month-old Non tg-SaCas9 (left), BAC226Q-SaCas9 (middle) and BAC226Q-HTTg1 mice (right) in the open field experiment, with darker colors representing longer time spent in that position and lighter colors representing shorter time spent in that position, the locomotor trajectory of Non tg-SaCas9 was mainly along the perimeter of the open field, the trajectory of BAC226Q-SaCas9 was mainly in one corner, and the trajectory of BAC226Q-HTTg1 was more dispersed than that of BAC226Q-SaCas9. FIG. 11B is the standard devation of ambulatory distance in four quadrants for 6-month-old mice, reflecting differences of time spend in the four quadrants (N=10 for each group). FIG. 11C is the total movement distance in the open field for 2.5-, 4-, and 6-month-old Non tg-SaCas9, BAC226Q-SaCas9, and BAC226Q-HTTg1 mice, with N=9 for each group at 2.5-and 4-month-old mice and N=10 for each group at 6-month-old mice; and FIG. 11D is the stereotypic counts for 2.5-, 4-, and 6-month-old Non tg-SaCas9, BAC226Q-SaCas9, and BAC226Q-HTTg1 mice in the open field (stereotypic counts), N=9 for each group in 2.5-month-old and 4-month-old mice, N=10 for each group in 6-month-old mice; from left to right are data for Non tg-SaCas9, BAC226Q-SaCas9 and BAC226Q-HTTg1. Data are analyzed by one-way ANOVA with Tukey's post hoc test, where *** indicates p<0.001, ** indicates p<0.01, * indicates p<0.05, and data are presented as mean±SEM.

FIG. 12 illustrates the weight of BAC226Q mice after SaCas9-HTTg1 treatment according to the embodiment of the present disclosure, with the weight changes of Non tg-SaCas9, BAC226Q-SaCas9 and BAC226Q-HTTg1 mice recorded from 0 to 30 weeks after virus injection; the number of mice in the Non tg-SaCas9 group at the initial time are 27, the number of mice in the BAC226Q-SaCas9 and BAC226Q-HTTg1 groups at the initial time are 26; the number of mice in the Non tg-SaCas9 group at the termination time are 6, the number of mice in the BAC226Q-SaCas9 group at the termination time are 3; and the number of mice in the BAC226Q-HTTg1 group at the termination time are 5; and data are analyzed by two-way ANOVA with Tukey's post hoc test, where ** indicates p<0.01, * indicates p<0.05, and data are presented as mean±SEM.

FIG. 13 illustrates the survival curve of BAC226Q mice after SaCas9-HTTg1 treatment according to the embodiment of the present disclosure, with the initial number of mice in the Non tg-SaCas9, BAC226Q-SaCas9 and BAC226Q-HTTg1 groups are 17, 17 and 21, respectively. Data are analyzed by log-rank (Mantel-Cox) test, and the p-value for the comparison of BAC226Q-SaCas9 and BAC226Q-HTTg1 groups is 0.0061.

FIGS. 14A-14C illustrates the phenotypic analysis of BAC226Q mice after AAV9-SaCas9-HTTg1 striatal injection according to the embodiment of the present disclosure, where FIG. 14A is the rota-rod test performed on 12-16 week old wild-type and BAC226Q mice after AAV-SaCas9-HTTg1 or AAV-SaCas9 injection, N=13. FIG. 14B is the open field test performed on 3-month-old, 3.5-month-old and 5-month-old wild-type and BAC226Q mice after AAV-SaCas9-HTTg1 or AAV-SaCas9 injection, respectively, N=8; and FIG. 14C is the weight of mice by one-month intervals, and there was no significant difference in the weight of BAC226Q-SaCas9 and BAC226Q-HTTg1 (Non tg-SaCas9, N=17-28; BAC226Q-SaCas9, N=15-27; BAC226Q-HTTg1, N=15-32). Data are analyzed by one-way ANOVA with Tukey's post hoc test, and n.s. indicates no significant difference, with data presented as mean±SEM.

FIGS. 15A-15C illustrates the editing efficiency and off-target effects of PEM-Seq detection of SaCas9-HTTg1 according to the embodiment of the present disclosure, with genomic DNA derived from brain striatum and cortex of 2-month-old and 13-month-old BAC226Q mice or BAC226Q primary cultured neurons infected with AAV-SaCas9-HTTg1, and BAC226Q primary cultured neurons uninfected with AAV-SaCas9-HTTg1 as negative controls, where FIG. 15A is the gene editing efficiency by PEM-Seq detection of SaCas9-HTTg1, and gene editing efficiency is the ratio of editing events to all events, with N=2 for the Neuron-Ctrl group, N=2 for Neuron-HTTg1 group, N=10 for 2 m BAC226Q-HTTg1 group, and N=4 for the 13 m BAC226Q-HTTg1 group. Data are shown as mean±SEM. FIG. 15B is the frequency of off-target events for SaCas9 detected by PEM-Seq, with N=2 for the Neuron-Ctrl group, N=2 for Neuron-HTTg1 group, N=10 for 2 m BAC226Q-HTTg1 group, and N=4 for the 13 m BAC226Q-HTTg1 group. Data are shown as mean±SEM; FIG. 15C is the Circos schematic of off-target loci detected by PEM-Seq, with arrows pointing to the loci of the human HTT gene and the off-target loci of the murine HTT gene, and curves connecting the targeting and off-target loci, with off-target causing chromosomal translocation between the two loci; black base sequences represent HTT sgRNA1 targeting loci, gray base sequences represent PAM sequences of SaCas9; and mismatches of human HTT and murine HTT are indicated by lowercase letters.

FIGS. 16A and 16B illustrates the editing products of the human mHTT gene according to the embodiment of the present disclosure, where FIG. 16A is the insertion-deletion form of human-derived mHTT cleavage sites detected by PEM-Seq, genome derived from the brains of 2-month-old and 13-month-old BAC226Q mice injected with AAV-SaCas9-HTTg1. Black base sequences represent HTT sgRNA 1 targeting sites, base sequences indicated by black underlines represent PAM sequences of SaCas9; base insertions are indicated by lowercase letters; base deletions are indicated by short horizontal lines; and vertical dashed lines represent the cleavage site of SaCas9 endonuclease; FIG. 16B is the relative frequency of the five major editing products of the human mHTT gene, and N=7.

### DETAILED DESCRIPTION OF THE EXEMPLARY EMBODIMENTS

In order to facilitate understanding of the present disclosure, a more comprehensive description of the disclosure is given below, and preferred embodiments of the disclosure are given. However, the present disclosure can be implemented in many different forms and is not limited to the embodiments described herein. Rather, these embodiments are provided for the purpose of providing a more thorough and comprehensive understanding of the present disclosure.

Unless otherwise defined, all technical and scientific terms used herein have the same meaning as commonly understood by those skilled in the art belonging to the present disclosure. The terms used herein in the specification of the present disclosure are for the purpose of describing specific embodiments only and are not intended to limit the disclosure.

As used herein, the term “and/or” includes any and all combinations of one or more of the relevant listed items.

An embodiment of the present disclosure for the sgRNA involves the nucleotide sequence as shown in SEQ ID NO: 1 (Guide Seq 1: 5′-TGGAAAAGCTGATGAAGGCCT-3′) or SEQ ID NO: 2 (Guide Seq 2: 5′-GAAGGCCTTCATCAGCTTTTC-3′); or nucleotide sequence; or a nucleotide sequence having at least 95% homology, e.g. 95% homology, 96% homology, 97% homology, 98% homology, 99% homology, and etc., and have the same function as the nucleotide sequence, or a nucleotide sequence obtained from the nucleotide sequence shown in SEQ ID NO: 1 or SEQ ID NO: 2 by deletion, substitution or addition of 1 to 6 bases, such as a nucleotide sequence by deletion, substitution or addition of 1, 2, 3, 4, 5 or 6 bases, and having the same function.

The present disclosure obtains the sgRNA targeting exon 1 of the human HTT gene with a nucleotide sequence as shown in SEQ ID NO: 1 or SEQ ID NO: 2 through research design and screening. The CRISPR/Cas9 system mediated HTT gene knockdown strategy based on this sgRNA and its higher homology sgRNA can efficiently knock down the human

Huntingtin gene for the gene therapy of Huntington's disease. In experimental tests, delivery of the AAV9 vector SaCas9 endonuclease and the aforementioned sgRNA targeting exon 1 of the human HTT gene into both striatal and cortical regions of the brain resulted in an approximately 90% reduction in HTT protein expression and, in particular, a significant improvement in HD-related phenotypic defects at the individual level. This gene therapy strategy has great potential to provide new ideas and technologies for the treatment of Huntington's disease.

In some specific examples, the nucleotide sequence of the sgRNA is a nucleotide sequence having at least 98% homology with the nucleotide sequence shown in SEQ ID NO: 1 or SEQ ID NO: 2 and having the same function.

In other specific examples, the nucleotide sequence of the sgRNA is a nucleotide sequence obtained from the nucleotide sequence shown in SEQ ID NO: 1 or SEQ ID NO: 2 by a 1 to 3 base deletion, substitution or addition at the 5′ end or 3′ end, and having the same function.

The DNA fragment of an embodiment of the present disclosure, which encodes the sgRNA as described above.

The recombinant expression vector of an embodiment of the present disclosure, the recombinant expression vector comprising a DNA fragment as described above. It will be appreciated that vector types include, but are not limited to: plasmids; phage particles; Coase plasmids; artificial chromosomes such as yeast artificial chromosomes (YAC), bacteriophage artificial chromosomes (BAC) or P1-derived artificial chromosomes (PAC); phages such as A-phages or M13-phages and animal viruses. Animal viruses that can be used as vectors include, but are not limited to, retroviruses (including lentiviruses), adenoviruses, adeno-associated viruses, herpes viruses (e.g., herpes simplex virus), pox viruses, baculoviruses, papillomaviruses, and papillary polyomavirus (e.g., SV40).

In one of the specific examples, the recombinant expression vector is an adeno-associated virus vector. Adeno-associated viruses are single-stranded DNA viruses that are effective gene therapy delivery vectors, capable of being present in the host cell as episome for long-term expression of the target gene. Adeno-associated viral vectors are available in different serotypes, each with different tissue and cellular tropism, and the common serotypes include AAV1, AAV2, AAV5, AAV6, AAV8 and AAV9, preferably AAV9. The AAV-CRISPR/Cas9 system can achieve efficient in vivo gene editing and has great potential for application. AAV9 has a high infection efficiency in the CNS, and is therefore well suited for use as a gene delivery vector in the treatment of CNS diseases.

In one of the specific examples, the recombinant expression vector also contains a

fragment of a Cas nuclease encoding sequence, such as, without limitation, SaCas9, SpCas9, Cas12a, Cas12b, Cas12e, Cas12j, Cas13a, Cas12f1 or Cas14a. Optionally, the Cas endonuclease coding sequence above is the SaCas9 endonuclease coding sequence. The use of the SaCas9 nuclease from Staphylococcus aureus allows SaCas9 and sgRNA to be packaged in the same adeno-associated viral vector, improving gene editing delivery efficiency. SaCas9 has almost the same level of gene editing efficiency as the most commonly used SpCas9, but the gene length of SaCas9 is 20% smaller than SpCas9 (SaCas9: 3.2 kb (1053 amino acids), SpCas9: 4.2 kb (1368 amino acids)). Since the packaging capacity of AAV9 can only be limited to 4.7 kb, only SaCas9 and sgRNA expression components can be assembled into the same AAV vector at the same time, and the simultaneous assembly of

Cas9 protein and sgRNA into an AAV viral vector can achieve more efficient in vivo delivery.

The virus of an embodiment of the present disclosure has a genome containing a nucleotide sequence encoding the sgRNA as described above. Virus types include, but are not limited to, retroviruses (including lentiviruses), adenoviruses, adeno-associated viruses, herpes viruses (e.g., herpes simplex virus), pox viruses, baculoviruses, papillomaviruses, papillary polyomavirus (e.g., SV40), and the like.

The present disclosure also provides a host cell having a genome containing a DNA fragment or recombinant expression vector as described above. Types of host cells include, but are not limited to, prokaryotic cells such as E. coli or Clostridium perfringens, fungal cells such as yeast cells or Aspergillus, insect cells such as S2 Drosophila cells or Sf9, or human cells such as fibroblasts, CHO cells, COS cells, NSO cells, HeLa cells, BHK cells or HEK293T cells, or animal cells.

The present disclosure also provides for the use of sgRNA, DNA fragments, recombinant expression vectors, viruses or host cells as described above in the preparation of products for the treatment of Huntington's disease.

In one of the specific examples, the products described above are reagents, kits, drugs or devices, etc. It is understood that the specific types are not limited thereto.

The drug for the treatment of Huntington's disease of an embodiment of the present disclosure, which comprises sgRNA, DNA fragments, recombinant expression vectors, viruses, or host cells, as described above, and pharmaceutically usable excipients.

In one of the specific examples, the dosage form of the above-described drug is an injection, but is not limited thereto.

In one of the specific examples, the excipients include one or more of a diluent, a preservative, a buffer, a disintegrant, an antioxidant, a co-suspension agent, a colorant, and an excipient.

In one of the specific examples, the diluent is selected from one or more of polyethylene glycol, propylene glycol, vegetable oil, and mineral oil. In a specific example, the preservative is selected from one or more of sorbic acid, methyl sorbate, methyl paraben, ethyl paraben, propyl paraben, butyl paraben, benzyl paraben, sodium methylparaben, benzoic acid, and benzyl alcohol. In another one of the specific examples, the buffering agent is selected from one or more of sodium hydrogen phosphate, sodium dihydrogen phosphate, sodium citrate, sodium tartrate, and sodium acetate. In another one of the specific examples, the disintegrant is selected from one or more of cross-linked sodium carboxymethyl cellulose, sodium carboxymethyl starch, cross-linked polyvinylpyrrolidone, or low-substituted hydroxypropyl cellulose. In another one of the specific examples, the antioxidant is selected from one or more of ethylenediaminetetraacetic acid, ethylenediaminetetraacetic acid disodium salt, dibutylhydroxytoluene, glycine, inositol, ascorbic acid, sodium ascorbate, lecithin, malic acid, hydroquinone, citric acid, succinic acid, and sodium metabisulfite. In another one of the specific examples, the co-suspension agent is selected from one or more of beeswax, ethyl hydroxyethyl cellulose, chitin, chitosan, methyl cellulose, carboxymethyl cellulose, agar, hydroxypropyl methyl cellulose, and xanthan gum. In another one of the specific examples, the colorant is selected from one or more of carbon black, iron black, iron brown, iron red, and titanium dioxide. In another one of the specific examples, the excipient is selected from one or more of mannitol, glucose, lactose, dextran, dextrose, and sodium chloride.

The HTT knockout method according to the embodiment of the present disclosure includes the steps of exogenously expressing the above sgRNA and Cas nucleic acid endonuclease in a cell. It will be appreciated that the HTT knockout method can be used for disease diagnostic and therapeutic purposes as well as for non-disease diagnostic and therapeutic purposes.

The method of treating Huntington's disease according to the embodiment of the present disclosure includes the steps of delivering the Cas nuclease system and the sgRNA as described above to the striatum and cortical regions of the brain of a diseased individual. Delivering both the sgRNA and the Cas endonuclease system into the striatum and cortex of the affected individual has been shown to be more effective in rescuing the disease phenotype in HD patients. Synergistic interactions between multiple brain regions, especially between the striatum and cortex, are critical for the rescue of the disease phenotype in HD patients.

In one of the specific examples, delivery is by brain stereotaxic injection, which allows precise and efficient delivery of the target gene to specific regions of the brain. In another one of the specific examples, the delivery approach is, without limitation, subarachnoid injection, lateral ventricular injection, cerebellar medullary pool injection, intravenous injection, and other delivery approaches.

The present disclosure will be described in further detail below in conjunction with specific embodiments and accompanying drawings.

### 1. Experimental Method

### 1.1 AAV-SaCas9-sgRNA Vector Construction

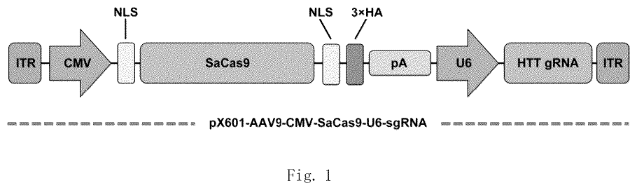

The single-stranded guide RNA is inserted into the AAV-Cas9 vector by Bsa I endonuclease digestion and ligation reactions, after annealing of the sense and antisense oligomeric strands of the phosphorylated single-stranded guide RNA to form a double-stranded structure. Thereafter, the resulting ligation product was transformed into Stbl3 competent cells and then plasmid DNA was isolated using the QIAprep spin miniprep kit according to the instructions. The sequence of the obtained DNA was verified by sequencing via LKO.1 5′ primer (LKO.1 5′ primer sequence: 5′-GACTATCATATGCTTACCGT-3′). The structure of the AAV-SaCas9-sgRNA vector is shown in FIG. 1. The vector backbone containing SaCas9 and sgRNA sequences is from Addgene (plasmid #61591), and the specific sequences of sgRNA and PAM are shown below:

| Guide Seq. | PAM |
|---|---|
| 5′-TGGAAAAGCTGATGAAGGCCT-3′ | 5′-TCGAGT-3′ |
| 5′-GAAGGCCTTCATCAGCTTTTC-3′ | 5′-CAGGGT-3′ |

### 1.2 HEK293T Cell Culture and Transfection

Human embryonic kidney (HEK) 293T cells (ATCC, CRL-1573) were cultured in DMEM medium (Dulbecco's modified Eagle's medium, Thermo Fisher) supplemented with 10% (v/v) fetal bovine serum (FBS, Thermo Fisher), 1% (v/v) penicillin and Streptomycin (Penicillin-Streptomycin, Thermo Fisher) and 1% (v/v) L-glutamine (Thermo Fisher), and the culture conditions were 37° C., 5% CO₂ environment. 293T cells were cultured to a density of about 70% and received 2.5 μg of AAV-SaCas9-sgRNA vector transfection, the kit used for transfection was lipofectamine 2000 (Invitrogen, 11668030), and 2.5 μg of pEGFPc3-human HTT exon 1-120Q or pEGFPc3-human HTT exon 1-20Q was transfected 16 hours later.

### 1.3 Purification of AAV9 Viral Vector

AAV vectors were amplified as previously reported (Ding et al., 2016; Grieger et al., 2006). Briefly, 10 plates of HEK293T cells were cultured to 90% density in a 15 cm diameter dish before transfection, and 5 mL of plasmid and PEI (polyethyleneimine, Sigma-Aldrich, 76509) mixture was added to each plate. The mixture contains 70 μg of AAV9 vector, 200 μg of Ad-Helper plasmid, 70 μg of AAV-Rep/Cap plasmid and PEI (1 μg/μL). The ratio of PEI and DNA used in this experiment is 5:1 (v/g). Add each reagent in the mixture into DMEM to a final volume of 50 mL and mix well for later use. 60 hours after transfection, the cells were collected with the medium and centrifuged at 1000 rpm for 10 minutes. Finally, the cell pellet was resuspended in 5 mL of cell lysis buffer (150 mM NaCl, 20 mM Tris, pH 8.0).

Cell lysates were freeze-thawed three times under conditions of liquid nitrogen and a 37° C. water bath to release the virus from the disrupted cell membranes. The cell lysate was added with a final concentration of 1 mM MgCl₂ and 250 U/mL nuclease Benzonase (Sigma, E8263-25k), and incubated at 37° C. for 15 min to dissolve DNA/protein aggregates. Afterwards, the cell lysate was centrifuged at 4000 rpm, 4° C. for 30 min and the supernatant was collected to obtain a virus solution.

The virus solution was added to the iodixanol gradient solution. The volume and density of the gradient solution from bottom to top were 6 mL 17% (5mL10×PBS, 0.05 mL 1M MgCl₂, 0.125 mL 1M KCl, 10 mL 5M NaCl, 12.5 mL Optiprep (Sigma, D1556), add water to 50 mL), 6 mL 25% (5 mL 10×PBS, 0.05 mL 1M MgCl₂, 0.125 mL 1M KCI, 20 mL Optiprep, add water to 50 mL), 5 mL 40% (5 mL 10×PBS, 0.05 mL 1M MgCl₂, 0.125 mL 1M KCI, 33.3 mL Optiprep, add water to 50 mL) and 4 mL 60% ((0.05 mL 1M MgCl₂, 0.125 mL 1M KCI, 50 mL Optiprep). Then the sample was centrifuged at 14° C., 53000 rpm for 160 minutes, and the virus were collected from the 40% layer.

The virus components were mixed with PBS solution and F188 (1:10000,Polomaxer, Sigma) and added to Amacon 100K filter (UFC910008, Millipore Sigma) and centrifuged at 4° C., 3500 rpm for 20 minutes to remove iodixanol and concentrate the virus. Afterwards, the filtrate was removed, and a PBS solution containing F188 was added to the virus fraction. The filter tubes were then centrifuged at 3500 rpm for 20 minutes at 4° C. Discard the filtrate again and obtain the virus fraction in PBS. The virus fraction was then centrifuged at 3500 rpm for 20 minutes at 4° C. until the virus volume was concentrated to 500 μL.

Virus titers can be determined by quantitative PCR. Afterwards, SDS-PAGE and Coomassie blue staining experiments were carried out to check the purity of the virus vector. In this experiment, the only proteins that could be observed on the SDS-PAGE gel were VP1, VP2 and VP3, which make up the capsid particles of virus, and the molecular weights were 87 kDa, 73 kDa and 62 kDa, respectively.

### 1.4 Stereotaxic Injection of Mouse Brain

In this example, the BAC226Q HD mouse model was used for preclinical proof-of-concept study. The BAC226Q mouse model was generated by transgenically expressing the human mHTT gene carrying 226 CAA-CAG mixed repeats in wild-type mouse embryos using Bacterial Artificial Chromosome (BAC). BAC226Q mice can express human mHTT protein with 226 glutamines. The expression of this protein causes BAC226Q mice to exhibit a series of HD patients related phenotypes, and BAC226Q mouse model is currently the only HD mouse model that can accurately recapitulate the disease phenotypes in Huntington's disease patients, and with no other phenotypes not present in patients.

AAV brain stereotaxic injection into BAC226Q mice or non-transgenic control mice were performed during the period of 26-30 days after birth. Mice were first anesthetized and then fixed on a stereotaxic apparatus (RWD, 68019). Afterwards, the scalp of mouse was disinfected with alcohol and povidone iodine, and the scalp was cut open to expose the skull, and then the skull was punched at a specific and appropriate coordinate position. The corresponding anterior-posterior (AP) and medial-lateral (ML) stereotaxic coordinates were calculated on the dural surface of the mouse. A total volume of 2.5 μL of AAV9 virus was injected into the striatum of mice at a rate of 0.3 μL/min (coordinates: +0.8 mm rostral to Bregma, +2.1 mm lateral to medial and −3.1 mm ventral from brain surface). A total volume of 0.5 μL was then injected into the mouse primary motor cortex at a rate of 0.1 L/min (coordinates: +1.5 mm rostral to Bregma, ±1.5 mm lateral to medial and −1.0 mm ventral from brain surface). The titer of AAV9 was 2×10¹³ viral genomes/mL, and it was injected on both sides, so the amount of virus injected per mouse brain was 1.2×10¹¹ viral genomes. The instrument used for virus injection was a Hamilton syringe connected to a microsyringe pump (RWD, 788130). The Hamilton syringe used for virus delivery was a 1701 Hamilton microsyringe (Hamilton, 7853-01) with a 33-gauge needle (Hamilton, 7803-05). Leave the needle in place for 15 minutes after each injection to reduce reflux of viral solution when the needle is withdrawn. After surgery the mice were placed on a heating blanket to recover from anesthesia.

### 1.5 Altered Expression of HTT Protein in Mouse Brains by Immunoprotein Blotting

Mice brain were dissected in an ice bath of PBS. Brain tissues were incubated in RIPA buffer (150 mM NaCl, 0.1% SDS, 1% sodium deoxycholate, 50 mM Triethanolamine, 1% NP-40, pH 7.4) with protease inhibitor cocktail (Sigma, 111M4009) and caspase inhibitor Boc-D-FMK (Abcam, ab142036), pH 7.4) and was lysed on ice using an ultrasonic shaker (Fisherbrand, FB120) at 20% sonication intensity, with a 2-second pause after 1 second of sonication, and repeated 20˜25 times. The lysis products were incubated for 1 h at 4° C. and then centrifuged at 16100 g for 20 min at 4° C. to remove insoluble fractions. The protein concentration in the lysate was determined by detecting the peak absorption at 562 nm with a BCA Protein Assay Kit (Thermo Scientific, 23225). Subsequently, NuPAGE 4×LDS sample buffer and 10× sample reducing agent (Invitrogen) were added to the lysate and heated at 70° C. for 10 min to complete the sample preparation. 90˜120 μg of protein was loaded to the 4% ˜12% NuPAGE Bis-Tris gel with MOPS running buffer. Protein blots were transferred to Immobilon-FL PVDF membranes (Millipore, IPFL00010) using the wet transfer method. The PVDF membranes with immunoblots were then treated in Odyssey blocking buffer (LI-COR, 927-40000) for 1 hour. After TBST rinsing, PVDF membranes are incubated overnight in primary antibody buffer diluted with blocking buffer at 4° C. PVDF membranes with immunoblots are then rinsed 3 times (10 min each) in TBST and incubated in IRDye 680RD goat anti-mouse (1:10,000, LI-COR, 926-68070) or goat anti-rabbit (1:10,000, LI-COR, 926-68071) secondary antibodies for 1 h at room temperature. The protein signal can be detected with the Odyssey CLx imager (LI-COR) at 700 nm channel conditions. The primary antibodies used in this example are shown below: 1C2 (1:5000, mouse, Millipore, MAB1574), β-actin (1:2000, rabbit, Cell Signaling, 4970) and GFAP (1:50000, rabbit, Abcam, ab7260).

### 1.6 Detection of HTT Protein Expression in Mouse Brain by Immunofluorescence and Immunohistochemical Staining

The experimental mice were perfused with 4% paraformaldehyde after anesthesia and then mouse brains were fixed in the same fixation solution overnight. The fixed brains were sliced at 30 μm or 40 μm thickness with a vibrating blade microtome (Leica, VT1200S). The brain sections were then blocked at room temperature for 1 h in PBS buffer supplemented with 10% sheep serum containing 0.1% to 0.3% Triton X-100, and incubated overnight at 4° C. with primary antibody solution, followed by washing off the antibody. The staining was continued by incubation in fluorescent secondary antibody for immunofluorescence staining. Alternatively, the sections were treated with biotinylated protein A and ABC peroxidase complex and incubated with diaminobenzidine, and the sections were mounted on slides for subsequent observation.

### 1.7 Behavioral Tests in Mice to Detect Treatment Effects

Behavioral analyses of mice were performed under the same environmental conditions, the same time conditions, and the same experimental staff. The genotypes of the mice and the experimental treatment conditions were not known at the time of the experiments.

Rota-rod experiments: Mice were trained on a rota-rod treadmill (Med Associates, Inc., ENV-574M) three times a day for three consecutive days. Mice were trained at a constant speed of 10 rpm/min for 1 min each time. During the training trials, mice dropped from the rota-rod were gently placed back. Mice were tested three times a day, three days a week, with a minimum of 15 minutes of rest between each trial. During testing, the rota-rod was accelerated from 5 rpm to 40 rpm in a maximum period of 300 seconds. The latency to fall from the rota-rod was recorded, and all data obtained from the experiments were statistically analyzed. The latency to fall was defined as the time it took for the mouse to fall off the rota-rod or the time it took for the mouse to hold the rod for more than three cycles. All rota-rod experiments were performed during the dark phase of the light cycle.

Open-field experiments: The mice used for the open-field experiments were 4 and 7 months of age. The experiments were performed according to the established experimental procedure over a 10-minute period. All experiments were performed at the same time of day. The apparatus for the open-field experiments consisted of a clear glass box (28×28 cm, Med Associates, Inc., ENV-510). Schematic showing the time and position of the mice in the cage was completed by R (https://www.r-project.org/), which represents the average data of 9 experiments per mouse (3 days, 3 times per day).

Gait analysis experiments: Mouse gait was analyzed using CatWalk XT (Noldus Information Technology, Wageningen, Netherlands) software. The CatWalk system consists of an enclosed walking channel mounted on a glass platform, from one end of which the mice traverse to the other during the experiments. A completely internally reflected green light is able to emitted only at areas where the paws make contact with the glass plate. A high-speed camera placed under the walkway records the paw prints. The image data from the captured paw prints will be used for footprint classification and subsequent analysis experiments. Prior to the experiments, the mice are trained for at least 4 days, at least 4 times per day, to adapt to the process of walking through a 70 cm long walkway using a non-forcible, intrusive method. The experiments were conducted in a dark room and silent conditions. At the day of experiment, mice were tested three times a day at the same time points for three consecutive days. Any behaviors that included wall climbing, hair grooming, and staying in the walkway were not statistically analyzed. Mice that did not successfully undergo CatWalk training were not counted in the experimental results. Each mouse used for gait analysis received an average of 3 to 6 compliant trials. The software used for analysis was CatWalk XT 10.6.

Body weight: After the surgery, mice were measured weekly for body weight and the results were displayed in grams.

Survival: The experimental mice were cultured in mixed genotypes with 3 to 5 mice per cage. Survival rates of mice were counted weekly after the procedure was performed.

### 1.8 Detection of Editing Efficiency and Off-Target Efficiency by Primer Extension Sequencing

AAV9-HTTg1-transduced cultured primary neurons and AAV9-HTTg1-injected mice (2 and 13 months old) brain samples were collected, washed with PBS, and digested with lysis buffer (50 Mm Tris-HCl, 50 mM EDTA, 1% SDS, 1% protease K). Genomic DNA was then extracted by phenol-chloroform extraction, and PEM libraries were prepared as previously reported (Yin et al., 2019). Genomic DNA was sonicated to 300-500 bp, and biotinylated CTCAGGTTCTGCTTTTACCTGCG sequences were used for primer extension experiments followed by bridge adapter ligation. The CCGAGGCCTCCGGGGACTGC sequence was used for nested PCR, followed by tagged PCR with primers compatible for Illumina Hiseq. Raw data processing has been previously reported, and gene editing efficiency is defined as the percentage of insertion-deletion events and translocation events to all events; and off-target rate is calculated as the percentage of off-target translocation events to total editing events.

### 2. Characterization Data and Effect Data of Examples and Comparative Examples

2.1 In Vitro Assays to Detect the Effect of CRISPR/Cas9 Editing on the mHTT Gene

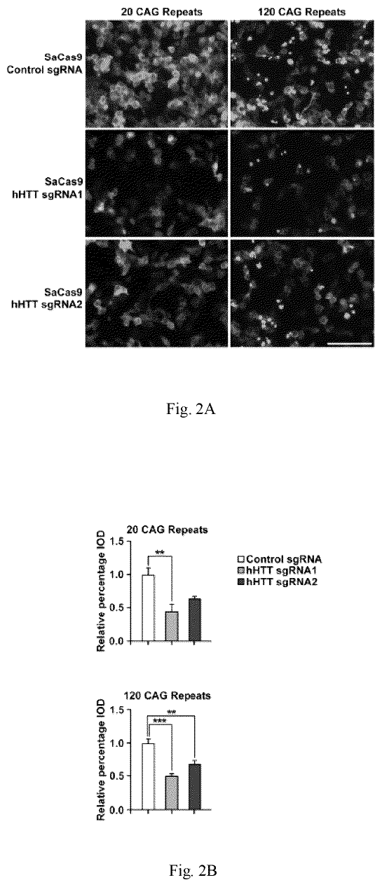

HEK293T cells were transfected with human HTT exon 1 containing 20 or 120 CAG repeats and fused with GFP protein sequences. When the GFP reporter is expressed together with the over-expanded 120Q, it forms intracellularly localized inclusions. The fusion expression of GFP protein with human HTT exon 1 allows for the detection of the cleavage efficiency of SaCas9-sgRNA. After transfection of HEK293T cells with either AAV-SaCas9-hHTT sgRNA1 (SaCas9-HTTg1) or AAV-SaCas9-hHTT sgRNA2 (SaCas9-HTTg2), GFP expression was significantly reduced in cells expressing either 20 or 120 CAG repeats, demonstrating that exon 1 of human HTT can be edited by the provided CRISPR/Cas9 system. There was a significant reduction in the number of human mutant HTT aggregates in cells co-expressing exon 1 with the CAG overexpanded sequence (FIG. 2A). The statistics further demonstrate that the number of aggregates in the transfected HTTg1 cells was reduced to 43.78% of that in the control group and 63.02% of that in the HTTg2 group under similar conditions of SaCas9 expression (FIG. 2B). The above in vitro results confirm that CRISPR/Cas9 system can achieve an efficient HTT gene editing.

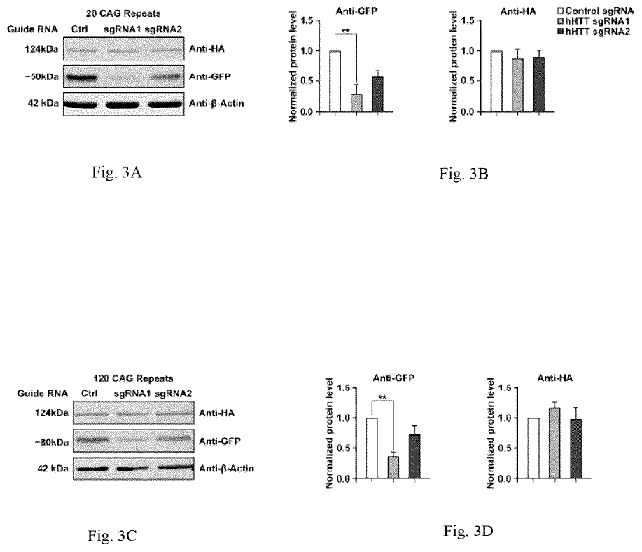

The expression of GFP and SaCas9 in HEK293T cells was also analyzed by immunoblotting. The results showed that in cells expressing GFP-HTT-exon1-20Q, transient transfection of sgRNA1 significantly reduced the expression level of GFP at the same level of SaCas9 expression, while sgRNA2 also reduced the expression level of GFP compared with the control group, but not as significantly as sgRNA1 (FIGS. 3A and 3B). The same results were observed in cells expressing GFP-HTT-exon1-120Q (FIGS. 3C and 3D). The above results reconfirmed the successful expression of SaCas9 in cells and the knockdown effect of HTT sgRNA1 on human HTT gene.

### 2.2 CRISPR/Cas9 Gene Editing in BAC226Q Mice

Taking HTT sgRNA1 as an example, to explore the role of the AAV9-SaCas9-sgRNA system in BAC226Q HD mice, and to comprehensively and profoundly assess the therapeutic effects of CRISPR/Cas9-mediated in vivo mHTT knockdown in terms of long-term pathology and disease phenotypes in mice.

### 2.2.1 Precise Infection of Mouse Striatum and Primary Motor Cortex With AAV9.

AAV9-GFP was injected into the striatum and primary motor cortex of 26-30 days

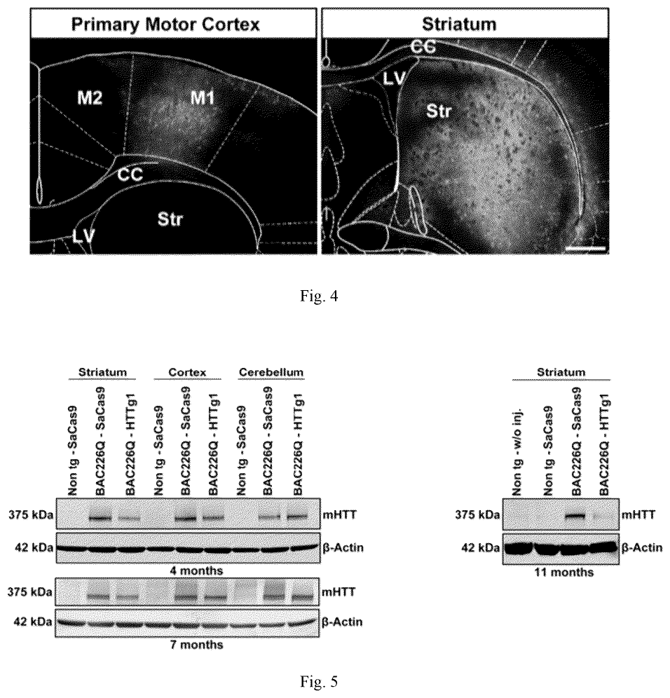

old non-transgenic mice by stereotaxic brain injection. One month after injection, brain sections of mice were collected for GFP expression analysis. Precise expression of GFP was observed in the striatum and primary motor cortex (FIG. 4), demonstrating the successful delivery of the AAV9 virus to the mouse brain and the stable expression of the genes contained therein.

2.2.2 SaCas9-HTTg1 Significantly Decreased mHTT Protein Expression in BAC226Q Mice

Striatal and cortical proteins were extracted from 4-, 7- and 11-month-old mice, respectively, and immunoblotting experiments were performed. These two brain regions received only one-time injection of AAV9-SaCas9-HTTg1 or AAV9-SaCas9 at 26 to 30 days of age, where BAC226Q mice injected with AAV9-SaCas9-HTTg1 were the experimental group (referred to as BAC226Q-HTTg1) and BAC226Q and wild-type mice injected with AAV9-SaCas9 were the control group (referred to as BAC226Q-SaCas9 and Non tg-SaCas9).

The expression of mHTT protein in the striatum of 4-month-old mice was significantly lower in BAC226Q-HTTg1 compared with the BAC226Q-SaCas9 group, and the same result was observed in the cortex. However, the expression of mHTT protein in BAC226Q-HTTg1 was not significantly altered in the cerebellar region that did not receive viral injection compared with BAC226Q-SaCas9. These results suggest that the sgRNA1-based AAV-CRISPR/Cas9 system can mediate the reduction of mHTT protein expression in the brains of BAC226Q mice. The same experiment was performed in 7-month-old mice, and immunoblotting results showed that mHTT protein expression was somewhat reduced in the striatum and cortex, but not in the cerebellum, in mice that received AAV-SaCas9-HTTg1 injection compared with BAC226Q mice that received AAV-SaCas9 injection. The mHTT expression in the striatum of 11-month-old mice was then examined, and the mHTT protein in the striatum of BAC226Q-HTTg1 mice was significantly reduced compared with the BAC226-SaCas9 control group (FIG. 5).

2.2.3 SaCas9-HTTg1 Continuously and Significantly Reduces mHTT Expression and Aggregation in the Brains of BAC226Q Mice

The most important pathological features in the brains of HD patients are the neuropil aggregates and nuclear inclusions composed of mutant HTT in the striatal and cortical neurons. mHTT aggregates appear in BAC226Q mice from 4 months of age and evolve into widely distributed protein inclusions as the disease progresses. Brain sections from these mice were immune-stained for S830, an antibody that recognizes exon 1 of mHTT and detects not only the soluble form of mHTT protein but also neuropil aggregates and nuclear inclusions with high specificity.

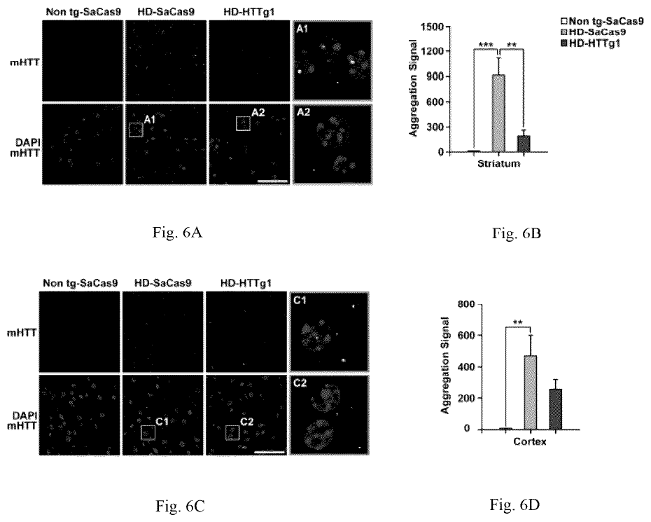

BAC226Q mice injected with AAV-SaCas9-HTTg1 (referred to as BAC226Q-SaCas9 or HD-SaCas9) had a significant reduction of mHTT aggregates as well as nuclear inclusions in the striatal region of the brain compared to BAC226Q mice receiving only AAV-SaCas9 injections (referred to as BAC226Q-SaCas9 or HD-SaCas9-HTTg1) in 4-month (FIGS. 6A and 6B). mHTT had a total signal of 0.6004±0.0612 in Non tg-SaCas9, 922.3±195.8 in BAC226Q-SaCas9 and 192.2±73.8 in BAC226Q-HTTg1. In the primary motor cortex, SaCas9-HTTg1 injection also reduced the mHTT aggregates and nuclear inclusions in 4-month-old BAC226Q mice (p=0.096). mHTT total signal was 0.1038+0.1038 in Non tg-SaCas9, 473.9±125.6 in BAC226Q-SaCas9, and 258.2±57.59 in BAC226Q-HTTg1 (FIGS. 6C and 6D).

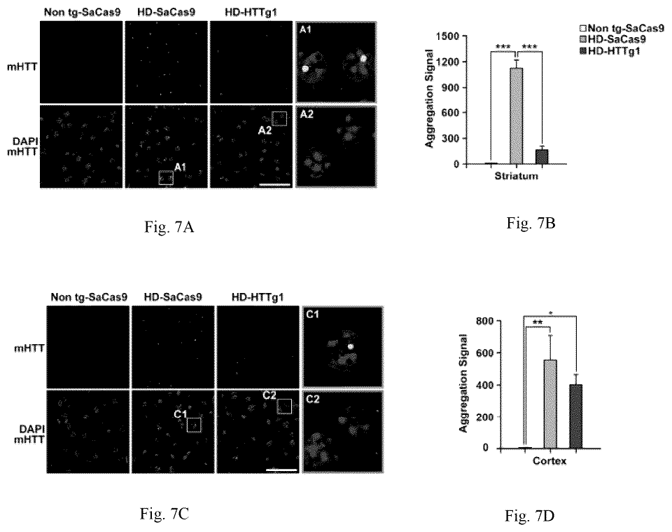

The mHTT in the striatum and cortex was mainly in the form of nuclear inclusions in 7-month-old mice compared with 4-month-old mice, with a more dense inclusion structure and a significant increase in size. In 7-month-old BAC226Q-SaCas9 mice, the signal of mHTT in the striatum and cortex was consistently increased, whereas the signal of mHTT in the striatum of BAC226Q-HTTg1 was consistently decreased. Compared with BAC226Q-SaCas9, the mHTT nuclear inclusion signal was significantly reduced in the striatum of 7-month-old BAC226Q-SaCas9-HTTg1 brain (FIGS. 7A and 7B). mHTT total signal was 0.0294±0.0294 in Non tg-SaCas9, 1129±87.26 in BAC226Q-SaCas9, and 168.2±35.26 in BAC226Q-HTTg1.

The mHTT signal in the primary motor cortex of BAC226Q-HTTg1 mice, although not reduced at a significant level (p=0.4982), was less in the form of nuclear inclusions and mHTT was mainly present in the form of aggregates. mHTT total signal was 0.6066±0.3189 in 7-month-old Non tg-SaCas9, 558.0±148.7 in BAC226Q-SaCas9, and 406.3±59.86 in BAC226Q-HTTg1 (FIGS. 7C and 7D).

The above experimental results indicated that the signal of aggregates and inclusions of mHTT in the brain of BAC226Q mice injected with AAV-SaCas9-HTTg1 was substantially reduced compared with BAC226Q-SaCas9 mice, and these signals are considered to be causative factors in the pathogenesis of HD, so the reduction of mHTT signal would be thought to improve the phenotype of the mice.

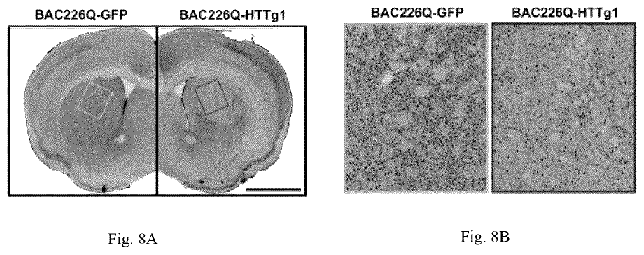

In addition, to test whether the in vivo clearance effect of mHTT by AAV9-CRISPR/Cas9 system would persist throughout the lifetime of the mice, immunohistochemical experiments were performed using brain section samples from 11-month-old mice to detect mutant HTT aggregates. Scans of whole brain sections showed a significant reduction of mHTT aggregates represented by S830 positive signals in the primary motor cortex and dorsal striatal region of BAC226Q mice injected with AAV-SaCas9-HTTg1 compared to controls injected with AAV-GFP only (FIGS. 8A and 8B). These results suggest that a single dose of CRISPR/Cas9 delivery can achieve a significant reduction of human mutant HTT aggregates in vivo across the lifespan.

### 2.2.4 SaCas9-HTTg1 Successfully Ameliorates Motor Deficits in BAC226Q Mice

BAC226Q mice exhibit early and strong Huntington's disease-like locomotor deficits, and these phenotypes develop progressively, with BAC226Q mice having normal locomotor function at 2 months of age but developing a severe hyperkinetic phenotype starting at 3 months of age. After that, the mice exhibit a reduced mobility phenotype after 7 months of age.

To examine whether CRISPR/Cas9-mediated human mutant HTT gene editing in vivo could rescue the disease phenotype in BAC226Q mice, this study focused on its effect on motor function. It was found that disruption of human mutant HTT expression by injecting AAV-SaCas9-HTTg1 into the striatum and primary motor cortex regions of BAC226Q mice significantly slowed the disease process at multiple disease-related time points.

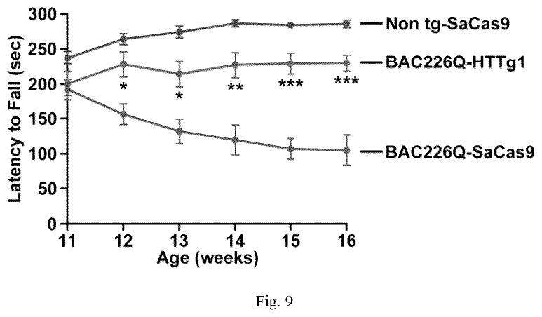

The rota-rod test is a widely accepted test for detecting movement-related phenotypes in Huntington's disease. It is often used to assess coordination and balance in mice. By measuring the time for mouse to fall from rota-rod, it was found that BAC226Q mice injected with AAV-SaCas9-HTTg1 into the striatum and primary motor cortex showed significant improvement and enhancement in coordination and locomotion ability early in the disease process (12-16 weeks of age) compared to mice injected with AAV-SaCas9 only (FIG. 9).

Another Huntington's disease phenotype that affects the quality of patient survival is abnormal walking behavior, so the gait phenotype of BAC226Q mice was assessed by the Catwalk gait analysis system, which records the walking behaviors of mice by photographing their behavior as they pass through the channel. Analysis of gait showed that 6-month-old

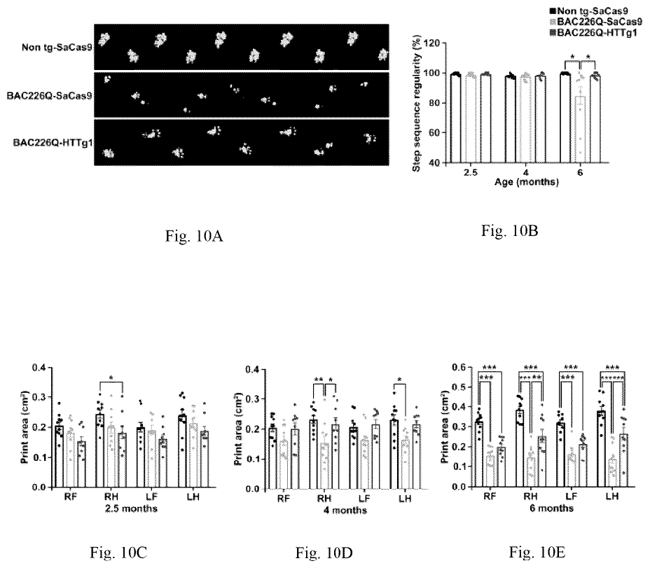

BAC226Q-SaCas9 mice exhibited a severe gait abnormality and defective phenotype compared with Non tg-SaCas9, which was restored in BAC226Q-HTTg1 mice, indicating that CRISPR/Cas9-mediated HTT knockdown rescued the gait phenotype of HD mice (FIG. 10A). The gait analysis revealed a progressive movement deficit phenotype in BAC226Q mice. During the gait analysis, the mice had six conventional gait patterns according to the order of using the left forelimb (LF), right forelimb (RF), left hindlimb (LH), and right hindlimb (RH): AA (RF-RH-LF-LH), AB (LF-RH-RF-LH), CA (RF-LF-RH-LH), CB (LF-RF-LH-RH), RA (RF-LF-LH-RH), and RB (LF-RF-RH-LH). The step sequence regularity index is used to reflect the regular gait pattern without the interference of missteps, which means that the more missteps are interspersed between the regular step patterns, the smaller the value of the step sequence regularity index. Therefore, this value is often used in gait analysis to reflect the degree of coordination in walking. The step sequence regularity of AAV-SaCas9-injected BAC226Q mice decreased with age, with a particularly significant decrease in 6-month-old BAC226Q-SaCas9 mice; however, AAV-SaCas9-HTTg1-injected BAC226Q mice maintained almost the same level of step sequence regularity as non-transgenic mice (FIG. 10B). Furthermore, by analyzing the size of the footprint area of the mice (FIGS. 10C, 10D, and 10E), the footprint area of BAC226Q mice from 2.5 to 4 months of age was progressively reduced compared to Non tg-SaCas9 control mice, and this reduction was more significant in 6-month-old mice. However, AAV-SaCas9-HTTg1 injections were effective in rescuing this disease phenotype in BAC226Q mice at 4 and 6 months of age. This demonstrates the rescue effect of motor performance in BAC226Q mice by human mutant HTT gene disruption.

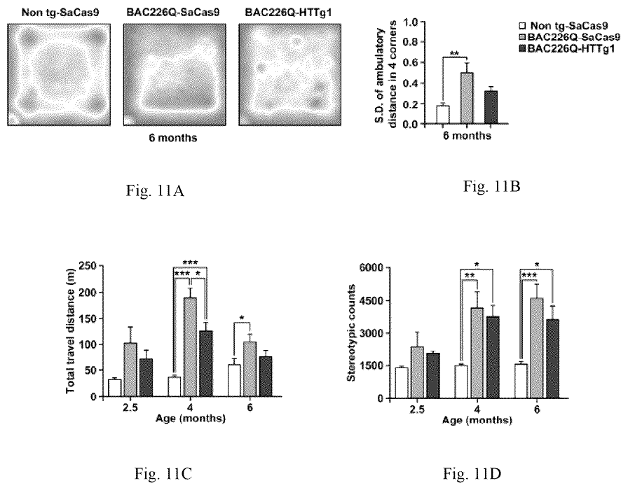

The next example explores whether the phenotype of BAC226Q-HTTg1 mice is rescued in the open field experiment. After 3 days of acclimatization in the open field for 10 min each time 3 times a day, the locomotor behavior of mice in the open field test was monitored for 3 consecutive days for 10 min each time 3 times a day, and the locomotor phenotype of mice in the open field was analyzed (FIGS. 11A-11D). The phenotype of the mice in the open field was more reflective of their spontaneous activity after a long period of acclimatization, and therefore could be better used to detect the phenotype of the mice in terms of locomotion. In 6-month-old mice, Non tg-SaCas9 mice tend to move more around the edge of the open field (Fig, 11A left); however, BAC226Q-SaCas9 mice tend to stay in a fixed corner of the open field and rotated (FIG. 11A middle); BAC226Q mice injected with AAV-SaCas9-HTTg1 have more opportunities to move around the open field (FIG. 11A right). The standard deviation of the distance moved in each quadrant was used as the statistical data. The smallest standard deviation of 0.1829 was found for Non tg-SaCas9, while the standard deviation of 0.5026 was found for BAC226Q-SaCas9 and 0.3295 for BAC226Q-HTTg1 (FIG. 11B), indicating that after SaCas9-HTTg1 expression, BAC226Q mice were more likely to move in the open field. BAC226Q mice had a more average locomotor area in the open field.

In addition, the total travel distance in the open-field experiment showed a decrease at 4 months of age and a slight decrease at 6 months of age for BAC226Q mice that received AAV-SaCas9-HTTg1 injection compared with BAC226Q mice that received only AAV-SaCas9 (FIG. 11C). For BAC226Q mice that received AAV-SaCas9-HTTg1 injection, stereotypic counts reflecting mouse rotation and stereotypic behaviors including self-grooming were also decreased (FIG. 11D). Combined with gait performance, these data suggest that AAV-SaCas9-HTTg1-mediated gene editing is able to slow down the motor deficits of BAC226Q mice.

### 2.2.5 SaCas9-HTTg1 Successfully Enhances Body Weight and Significantly Improves Mouse Survival in BAC226Q Mice

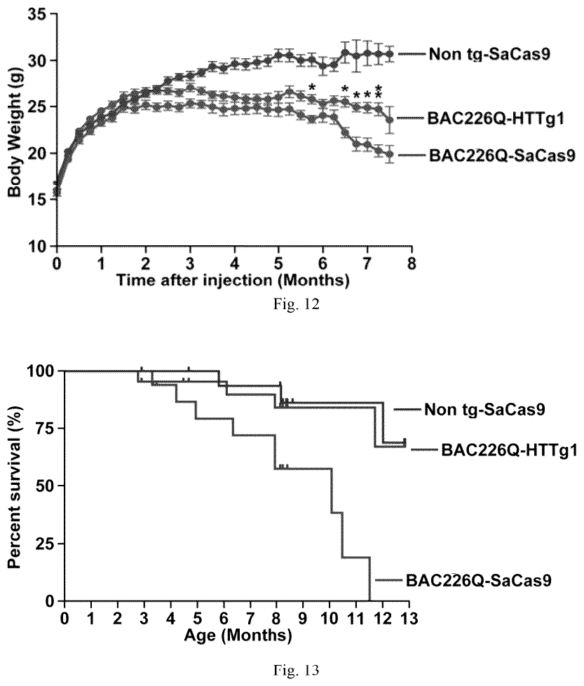

BAC226Q mice exhibit disease phenotypes similar to those of HD patients, including weight loss and shortened life span, making this HD mouse model highly useful for assessing the long-term therapeutic effects of candidate treatment in survival quality. In this study, body weights of mice after gene therapy were recorded weekly for a total of 0 to 30 weeks after viral injection (FIG. 12). The weight loss phenotype was attenuated in BAC226Q mice receiving AAV-SaCas9-HTTg1 injection compared to mice receiving AAV-SaCas9 injection in the striatum and primary motor cortex regions (FIG. 12).

The survival rate of the mice was continuously followed throughout their lifespan and was also significantly improved in BAC226Q mice receiving AAV-SaCas9-HTTg1 injection compared to BAC226Q-SaCas9 mice (FIG. 13) (p=0.0061). At the endpoint of this study (day 385), the survival rate of non-transgenic mice receiving AAV-SaCas9 injection and BAC226Q mice receiving AAV-SaCas9-HTTg1 injection remained above 50%; however, all BAC226Q mice that received AAV-SaCas9 injection died by the end of the study, and the median survival time of these BAC226Q mice was 302 days. In conclusion, these data suggest that CRISPR/Cas9-mediated human HTT knockout can successfully alleviate the HD-related phenotypes in BAC226Q mice.

### 2.2.6 Phenotypic Assay of BAC226Q Mice After AAV9-SaCas9-HTTg1 Striatal Injection

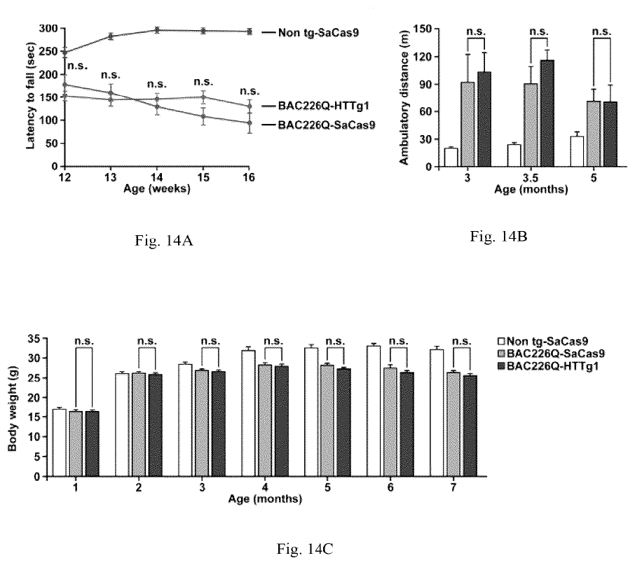

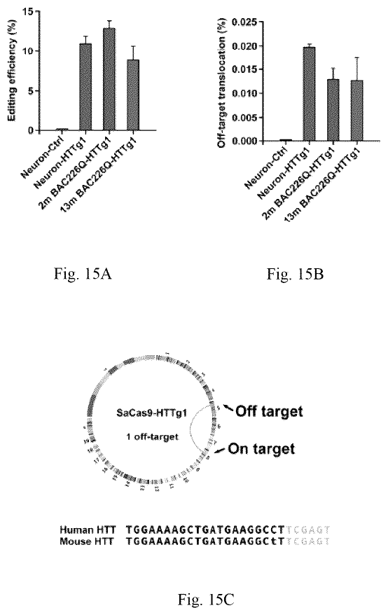

Preliminary results showed that when the CRISPR/Cas9 system was injected into the striatum only, the motor deficits in BAC226Q were not significantly improved from the rota-rod and open field experiments, despite the reduction in both mHTT protein and aggregates in the striatal region (FIGS. 14A and 14B), and the reduction in body weight of the mice was not significantly improved (FIG. 14C). Therefore, it indicates that targeting both striatal and cortical brain regions can better treat HD.

### 2.2.7 PEM-Seq Assay for Editing Efficiency and Off-Target Rate of AAV9-SaCas9-HTTg1 System

To investigate the editing efficiency and specificity of CRISPR/Cas9 in BAC226Q mice, PEM-Seq (primer-extension-mediated sequencing), a well-established deep sequencing method, was used to detect the editing events of CRISPR/Cas9 system, including indels, large deletions and genome-wide translocations. It can be used to simultaneously determine gene editing efficiency, off-target hotspots and gene editing products.

Genomic DNA was extracted from the brain striatum and cortex of BAC226Q mice injected with AAV-SaCas9-HTTg1 and from Cas9-HTTg1-treated BAC226Q mouse primary cultured neurons, and PEM-Seq experiments were performed to analyze editing events. Editing efficiency is 11.0%±0.9% in BAC226Q primary cultured neurons detected by PEM-Seq, and is 12.94%±0.91% and 8.90%±1.75% in the brains of 2-month-old and 13-month-old BAC226Q mice, respectively (FIG. 15A). The above results not only demonstrated that the mHTT gene editing events existed in the brains of BAC226Q mice, but also demonstrated that the results of HTT gene editing in the brains of BAC226Q mice could persist throughout the life span, which was consistent with the results observed in immunoblotting experiments and immunofluorescence staining assays.

PEM-Seq can be used to detect all insertion, deletion and translocation events during gene editing. The presence of off-target junctions causes chromosomal translocations between target and off-target junctions, which can be detected by PEM-Seq. Off-target translocation efficiency is the ratio of off-target junctions to all gene editing events (insertions, deletions and chromosomal translocations). In primary cultured neurons, only 0.0198% +0.0006% of off-target translocation events were detected, and the off-target efficiency was 0.0130%±0.0022% in 2-month-old BAC226Q-SaCas9 mouse brains and 0.0127%±0.0047% in 13-month-old brains (FIG. 15B), a result that indicates the very low off-target efficiency of the SaCas9-HTTg1 system. Further analysis of the off-target sites did not detect any other off-target cleavage events except for the murine HTT gene, and the presence of this off-target site in the murine HTT gene was also due to sequence homology between the murine HTT gene and the human HTT gene in the target site region of the sgRNA (FIG. 15 C).

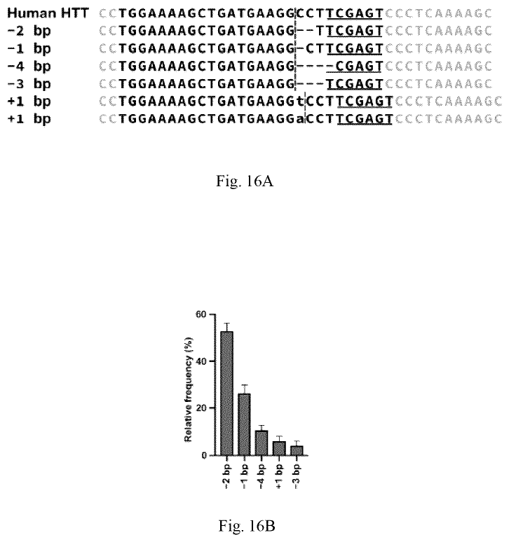

Analysis of the form of the gene editing products revealed that the editing events at the target sites of human HTT mainly showed small insertions and deletions (FIG. 16A). The 2 bp deletion and 1 bp insertion result in a change from glutamine to alanine and threonine and an early stop codon in exon 1; the 1 bp and 4 bp deletions result in a change from glutamine to serine and aspartate and an early stop codon in exon 2; and the 3 bp deletion does not result in a frameshift mutation. The relative frequencies of the five major editing products in the above gene editing were analyzed, with 52.70%±3.71% of the edited products are 2 bp deletion and only 4.126%±1.959% of the edited products are 3 bp deletion without a frameshift mutation (FIG. 16B). Taken together, these data demonstrate the editing effect of SaCas9-HTTg1 at the human HTT locus with a predictable off-target editing effect at the murine HTT locus.

In addition, sgRNA2 has also been experimentally tested to have similar effects to sgRNA1 as described above, but slightly less than sgRNA1.

Each technical feature of the above described embodiments can be combined in any way, and for the sake of concise description, not all possible combinations of each technical feature of the above described embodiments have been described. However, as long as the combinations of these technical features are not contradictory, they should be considered to be within the scope of the present specification.

The above described embodiments express only several embodiments of the present disclosure with more specific and detailed descriptions, but they are not to be construed as a limitation of the scope of the patent in the present disclosure. It should be noted that for a person of ordinary skill in the art, a number of deformations and improvements can be made without departing from the conception of the present disclosure, which all belong to the scope of protection of the present disclosure. Therefore, the scope of protection of the patent of the present disclosure shall be subject to the attached claims.

1-22. (canceled)

23. A recombinant expression vector comprising:
(a) a first DNA fragment encoding an sgRNA; wherein the sgRNA targets exon 1 of the human HTT gene, and the sgRNA comprises (i) the nucleotide sequence of SEQ ID NO: 1 (UGGAAAAGCUGAUGAAGGCCU) or (ii)
the nucleotide sequence of SEQ ID NO: 1 having substitution of one, two, or three bases; and
(b) a second DNA fragment encoding a Cas9 endonuclease.

24-25. (canceled)

26. A host cell comprising the recombinant expression vector according to claim 23.

27. (canceled)

28. The recombinant expression vector according to claim 23, wherein
(a) the sgRNA comprises the nucleotide sequence of SEQ ID NO: 1;
(b) the Cas9 endonuclease is a SaCas9 endonuclease or a SpCas9 endonuclease; or
(c) both (a) and (b).

29. The recombinant expression vector according to claim 23, wherein the recombinant expression vector is a lentiviral vector, an adenoviral vector, an adeno-associated virus vector, a herpesvirus vector, a poxvirus vector, a baculovirus vector, a papillomavirus vector, or a papillary polyomavirus vector.

30. The recombinant expression vector according to claim 23, wherein the recombinant expression vector is an AAV9 virus vector.

31. The host cell according to claim 26, wherein the host cell is a CHO cell, COS cell, NSO cell. HeLa cell, BHK cell or HEK293T cell.

32-62. (canceled)

63. The recombinant expression vector according to claim 28, wherein the Cas9 endonuclease is a SaCas9 endonuclease.

64. A drug for the treatment of Huntington's disease comprising the recombinant expression vector according to claim 23 and a pharmaceutically usable excipient.

65. The drug for the treatment of Huntington's disease according to claim 64, wherein the pharmaceutically usable excipient comprises one or more of diluents, preservatives, buffers, disintegrants, antioxidants, suspending agents, and colorants.

66. A method for the treatment of Huntington's disease, the method comprising: delivering the drug according to claim 64 to an individual affected by Huntington's disease.

67. The method for the treatment of Huntington's disease according to claim 66, wherein the delivering comprising stereotaxic injection of the drug into both the striatum and the cortex of the brain of the individual.

68. A method for producing the recombinant expression vector according to claim 23, the method comprising:
constructing the recombinant expression vector;
transfecting a host cell with the recombinant expression vector to produce a transfected host cell;
culturing the transfected host cell to produce cultured transfected host cells;
lysing the cultured transfected host cells to produce a cell lysate; and
purifying the recombinant expression vector from the cell lysate

69. A drug for the treatment of Huntington's disease comprising:
(a) a first DNA fragment encoding an sgRNA; wherein the sgRNA targets exon 1 of the human HTT gene, and the sgRNA comprises (i) the nucleotide sequence of SEQ ID NO: 1 (UGGAAAAGCUGAUGAAGGCCU) or (ii) the nucleotide sequence of SEQ ID NO: 1 having substitution of one, two, or three bases;
(b) a second DNA fragment encoding a Cas9 endonuclease; and
(c) a pharmaceutically usable excipient.

70. The drug according to claim 69, wherein
(a) the sgRNA comprises the nucleotide sequence of SEQ ID NO: 1;
(b) the Cas9 endonuclease is a SaCas9 endonuclease or a SpCas9 endonuclease; or
(c) both (a) and (b).

71. The drug according to claim 69, wherein the pharmaceutically usable excipient comprises one or more of diluents, preservatives, buffers, disintegrants, antioxidants, suspending agents, and colorants.

72. A method for the treatment of Huntington's disease, the method comprising: delivering the drug according to claim 69 to an individual affected by Huntington's disease.

73. The method for the treatment of Huntington's disease according to claim 72, wherein the delivering comprising stereotaxic injection of the drug into both the striatum and the cortex of the brain of the individual.

74. A method for the treatment of Huntington's disease, the method comprising:
delivering (a) a first DNA fragment encoding an sgRNA; wherein the sgRNA targets exon 1 of the human HTT gene, and the sgRNA comprises (i) the nucleotide sequence of SEQ ID NO: 1 (UGGAAAAGCUGAUGAAGGCCU) or (ii) the nucleotide sequence of SEQ ID NO: 1 having substitution of one, two, or three bases; and (b) a second DNA fragment encoding a Cas9 endonuclease to an individual affected by Huntington's disease.

75. The method for the treatment of Huntington's disease according to claim 74, wherein
(a) the sgRNA comprises the nucleotide sequence of SEQ ID NO: 1;
(b) the Cas9 endonuclease is a SaCas9 endonuclease or a SpCas9 endonuclease; or
(c) both (a) and (b).

76. The method for the treatment of Huntington's disease according to claim 74, wherein the delivering comprises stereotaxic injection of the first DNA fragment and the second DNA fragment into both the striatum and the cortex of the brain of the individual.


SEQUENCE LISTING (14 sequences)

SEQ ID NO: 1 - RNA, 21 nt, Homo sapiens
  source 1..21: mol_type = other RNA
  ncRNA 1..21: ncRNA_class = sgRNA
tggaaaagctgatgaaggcct

SEQ ID NO: 2 - RNA, 21 nt, Homo sapiens
  source 1..21: mol_type = other RNA
  ncRNA 1..21: ncRNA_class = sgRNA
gaaggccttcatcagcttttc

SEQ ID NO: 3 - DNA, 20 bp, synthetic construct
  source 1..20: mol_type = other DNA
gactatcatatgcttaccgt

SEQ ID NO: 4 - DNA, 23 bp, synthetic construct
  source 1..23: mol_type = other DNA
ctcaggttctgcttttacctgcg

SEQ ID NO: 5 - DNA, 20 bp, synthetic construct
  source 1..20: mol_type = other DNA
ccgaggcctccggggactgc

SEQ ID NO: 6 - DNA, 27 bp, Homo sapiens
  source 1..27: mol_type = other DNA
tggaaaagctgatgaaggccttcgagt

SEQ ID NO: 7 - DNA, 27 bp, Mus musculus
  source 1..27: mol_type = other DNA
tggaaaagctgatgaaggctttcgagt

SEQ ID NO: 8 - DNA, 40 bp, Homo sapiens
  source 1..40: mol_type = other DNA
cctggaaaagctgatgaaggccttcgagtccctcaaaagc

SEQ ID NO: 9 - DNA, 38 bp, Homo sapiens
  source 1..38: mol_type = other DNA
cctggaaaagctgatgaaggttcgagtccctcaaaagc

SEQ ID NO: 10 - DNA, 39 bp, Homo sapiens
  source 1..39: mol_type = other DNA
cctggaaaagctgatgaaggcttcgagtccctcaaaagc

SEQ ID NO: 11 - DNA, 36 bp, Homo sapiens
  source 1..36: mol_type = other DNA
cctggaaaagctgatgaaggcgagtccctcaaaagc

SEQ ID NO: 12 - DNA, 37 bp, Homo sapiens
  source 1..37: mol_type = other DNA
cctggaaaagctgatgaaggtcgagtccctcaaaagc

SEQ ID NO: 13 - DNA, 41 bp, Homo sapiens
  source 1..41: mol_type = other DNA
cctggaaaagctgatgaaggtccttcgagtccctcaaaagc

SEQ ID NO: 14 - DNA, 41 bp, Homo sapiens
  source 1..41: mol_type = other DNA
cctggaaaagctgatgaaggaccttcgagtccctcaaaagc